SEQUENCE LISTING (14 sequences)

SEQ ID NO: 1 - protein, 712 aa, Homo sapiens
MDHAEENEILAATQRYYVERPIFSHPVLQERLHTKDKVPDSIADKLKQAFTCTPKKIRNIIYMFLPITKWLPAYKFKEYVLGDLVSGISTGVLQLPQGLAFAMLAAVPPIFGLYSSFYPVIMYCFLGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLKESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQLAGCLASLMILLVILATGFLFESLPQTIWLTTFVSSLFLGLDYGLITAVIIALLTVIYRTQSPSYKVLGKLPETDVYIDIDAYEEVKEIPGIKIFQINAPIYYANSDLYSNALKRKTGVNPAVIMGARRKAMRKYAKEVGNANMANATVVKADAEVDGEDATKPEEEDGEVKYPPIVIKSTFPEEMQRFMPPGDNVHTVILDFTQVNFIDSVGVKTLAGIVKEYGDVGIYVYLAGCSAQVVNDLTRNRFFENPALWELLFHSIHDAVLGSQLREALAEQEASAPPSQEDLEPNATPATPEA

SEQ ID NO: 2 - protein, 744 aa, Mus musculus
MDHAEENEIPAETQRYYVERPIFSHPVLQERLHVKDKVTESIGDKLKQAFTCTPKKIRNIIYMFLPITKWLPAYKFKEYVLGDLVSGISTGVLQLPQGLAFAMLAAVPPVFGLYSSFYPVIMYCFFGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLHESYSVDVVGTLPLGLLPPANPDTSLFHLVYVDAIAIAIVGFSVTISMAKTLANKHGYQVDGNQELIALGICNSIGSLFQTFSISCSLSRSLVQEGTGGKTQLAGCLASLMILLVILATGFLFESLPQAVLSAIVIVNLKGMFMQFSDLPFFWRTSKIELTIWLTTFVSSLFLGLDYGLITAVIIALLTVIYRTQSPSYKVLGQLPDTDVYIDIDAYEEVKEIPGIKIFQINAPIYYANSDLYSSALKRKTGVNPALIMGARRKAMRKYAKEVGNANVANATVVKVDAEVDGENATKPEEEDDEVKFPPIVIKTTFPEELQRFLPQGENVHTVILDFTQVNFVDSVGVKTLAGIVKEYGDVGIYVYLAGCSPQVVNDLTRNNFFENPALKELLFHSIHDAVLGSQVREAMAEQEATASLPQEDMEPNATPTTPEA

SEQ ID NO: 3 - protein, 740 aa, Pteropus vampyrus
MDHAEENEILAATQRYYVERPIFSHPVLQERLHKKDKIPDSIGDKLKQAFTCTPKKIRNIIYMFLPITKWLPAYKFKEYVLGDLVSGISTGVLQLPQGLAFAMLAAVPPVFGLYSSFYPVIMYCFFGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLHESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQLAGCLASLMILMVILATGFLFESLPQAVLSAIVIVNLKGMFMQFSDLPFFWRTSKIELTIWLTTFVSSLFLGLDYGLITAVIIALMTVIYRTQSPSYKVLGQLPDTDVYIDIDAYEEVKEIPGIKIFQINAPIYYANSDLYSNALKRKTGVNPALIMGARRKAMRKYAKEVGNANMANAAVVKADAEVDGEDGTKPEEEDEVKYPPIVIKNTFPEELQRFMPPGDNIHTVILDFTQVNFIDSVGVKTLAGIVKEYGDVGIYVYLAGCSAQVVNDLTQNQFFENPALRELLFHSIHDAVLGSHVREALAEQEASAPATQEDMEPNATPEA

SEQ ID NO: 4 - protein, 741 aa, Balaenoptera acutorostrata
MDHAEENEILAAAQRYYVERPIFSHLVLQERLHKKDKISDSIGDKLKQAFTCTPKKIRNIIYMFLPITKWLPAYRFKEYVLGDLVSGISTGVLQLPQGLAFAMLAAVPPVFGLYSSFYPVIMYCFFGTSRHISIGPFAVISLMIGGVAVRLVPDDMVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLNESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQLAGCLASLMILLVILATGFLFESLPQAVLSAIVIVNLKGMFMQFSDLPFFWRTSKIELTIWLTTFVSSLFLGLDYGLITAVIIALLTVIYRTQSPSYKVLGQLPDTDVYIDIDAYEEVKEIPGIKIFQINAPIYYANSDLYSSALKRKTGVNPALIMGARRKAMKKYAKEVGNANMANATIVKVDAEVDGEDGTKPEEEENEIKYPPVVIKSTLPEELQRFMPPGDNVHTIILDFTQVNFIDSVGVKTLAGIVKEYGDVGIYVYLAGCSAQVVNDLSRNQFFENPALLELLFHSIHDAVLGSQVREALAEQEATAAPPQEDSEPNATPEA

SEQ ID NO: 5 - protein, 740 aa, Eonycteris spelaea
MDHAEENEILAATQRYYVERPIFSHPVLQERLHKKDKIPDSIGDKLKQAFTCTPKKIRNIIYMFLPITKWLPAYKFKEYVLGDLVSGISTGVLQLPQGLAFAMLAAVPPVFGLYSSFYPVIMYCFFGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLHESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQLAGCLASLMILMVILATGFLFESLPQAVLSAIVIVNLKGMFMQFSDLPFFWRTSKIELTIWLTTFVSSLFLGLDYGLITAVIIALMTVIYRTQSPSYKVLGQLPDTDVYIDIDAYEEVKEIPGIKIFQINAPIYYANSDLYSNALKRKTGVNPALIMGARRKAMRKYAKEVGNANMANAAVIKADAEVDGDDGTKPEEEDEVKYPPIVIKNTFPEELQRFMPPGDNVHTVILDFTQVNFIDSVGVKTLAGIVKEYGDVGIYVYLAGCSAQVVNDLTQNQFFENPALRELLFHSIHDAVLGSHVREALAEQEASAPTTQEDLEPNATPEA

SEQ ID NO: 6 - protein, 740 aa, Rousettus leschenaultia
MDHAEENEILAATQRYYVERPIFSHPVLQERLHKKDKIPDSIGDKLKQAFTCTPKKIRNIIYMFLPITKWLPAYKFKEYVLGDLVSGISTGVLQLPQGLAFAMLAAVPPVFGLYSSFYPVIMYCFFGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLHESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQLAGCLASLMILMVILATGFLFESLPQAVLSAIVIVNLKGMFMQFSDLPFFWRTSKIELTIWLTTFVSSLFLGLDYGLITAVIIALLTVIYRTQSPSYKVLGQLPDTDVYIDIDAYEEVKEIPGIKIFQINAPIYYANSDLYSNALKRKTGVNPAFIMGARRKAMRKYAKEVGNANMANAAVIKADAEVDGEDGTKPEEEDEVKYPPIVIKNTFPEELQRFMPPGDNIHTVILDFTQVNFIDSVGVKTLAGIVKEYGDVGIYVYLAGCSAQVVNDLTQNQFFENPALRELLFHSIHDAVLGSHVREALAEQEASAPTTQEDLEPNATPEA

SEQ ID NO: 7 - protein, 712 aa, artificial sequence
  synthetic sequence
MDHAEENEILAATQRYYVERPIFSHPVLQERLHTKDKVPDSIADKLKQAFTCTPKKIRNIIYMFLPITKWLPAYKFKEYVLGDLVSGISTGVLQLPQGLAFAMLAAVPPIFGLYSSFYPVIMYCFLGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFSLKESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQLAGCLASLMILLVILATGFLFESLPQTIWLTTFVSSLFLGLDYGLITAVIIALLTVIYRTQSPSYKVLGKLPETDVYIDIDAYEEVKEIPGIKIFQINAPIYYANSDLYSNALKRKTGVNPAVIMGARRKAMRKYAKEVGNANMANATVVKADAEVDGEDATKPEEEDGEVKYPPIVIKSTFPEEMQRFMPPGDNVHTVILDFTQVNFIDSVGVKTLAGIVKEYGDVGIYVYLAGCSAQVVNDLTRNRFFENPALWELLFHSIHDAVLGSQLREALAEQEASAPPSQEDLEPNATPATPEA

SEQ ID NO: 8 - protein, 712 aa, artificial sequence
  synthetic sequence
MDHAEETEILAATQRYYVERPIFSHPVLQERLHTKDKVPDSIADKLKQAFTCTPKKIRNIIYMFLPITKWLPAYKFKEYVLGDLVSGISTGVLQLPQGLAFAMLAAVPPIFGLYSSFYPVIMYCFLGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFSLKESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQLAGCLASLMILLVILATGFLFESLPQTIWLTTFVSSLFLGLDYGLITAVIIALLTVIYRTQSPSYKVLGKLPETDVYIDIDAYEEVKEIPGIKIFQINAPIYYANSDLYSNALKRKTGVNPAVIMGARRKAMRKYAKEVGNANMANATVVKADAEVDGEDATKPEEEDGEVKYPPIVIKSTFPEEMQRFMPPGDNVHTVILDFTQVNFIDSVGVKTLAGIVKEYGDVGIYVYLAGCSAQVVNDLTRNRFFENPALWELLFHSIHDAVLGSQLREALAEQEASAPPSQEDLEPNATPATPEA

SEQ ID NO: 9 - protein, 744 aa, artificial sequence
  synthetic sequence
MDHAEENEIPAETQRYYVERPIFSHPVLQERLHVKDKVTESIGDKLKQAFTCTPKKIRNIIYMFLPITKWLPAYKFKEYVLGDLVSGISTGVLQLPQGLAFAMLAAVPPVFGLYSSFYPVIMYCFFGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFSLHESYSVDVVGTLPLGLLPPANPDTSLFHLVYVDAIAIAIVGFSVTISMAKTLANKHGYQVDGNQELIALGICNSIGSLFQTFSISCSLSRSLVQEGTGGKTQLAGCLASLMILLVILATGFLFESLPQAVLSAIVIVNLKGMFMQFSDLPFFWRTSKIELTIWLTTFVSSLFLGLDYGLITAVIIALLTVIYRTQSPSYKVLGQLPDTDVYIDIDAYEEVKEIPGIKIFQINAPIYYANSDLYSSALKRKTGVNPALIMGARRKAMRKYAKEVGNANVANATVVKVDAEVDGENATKPEEEDDEVKFPPIVIKTTFPEELQRFLPQGENVHTVILDFTQVNFVDSVGVKTLAGIVKEYGDVGIYVYLAGCSPQVVNDLTRNNFFENPALKELLFHSIHDAVLGSQVREAMAEQEATASLPQEDMEPNATPTTPEA

SEQ ID NO: 10 - protein, 744 aa, artificial sequence
  synthetic sequence
MDHAEETEIPAETQRYYVERPIFSHPVLQERLHVKDKVTESIGDKLKQAFTCTPKKIRNIIYMFLPITKWLPAYKFKEYVLGDLVSGISTGVLQLPQGLAFAMLAAVPPVFGLYSSFYPVIMYCFFGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFSLHESYSVDVVGTLPLGLLPPANPDTSLFHLVYVDAIAIAIVGFSVTISMAKTLANKHGYQVDGNQELIALGICNSIGSLFQTFSISCSLSRSLVQEGTGGKTQLAGCLASLMILLVILATGFLFESLPQAVLSAIVIVNLKGMFMQFSDLPFFWRTSKIELTIWLTTFVSSLFLGLDYGLITAVIIALLTVIYRTQSPSYKVLGQLPDTDVYIDIDAYEEVKEIPGIKIFQINAPIYYANSDLYSSALKRKTGVNPALIMGARRKAMRKYAKEVGNANVANATVVKVDAEVDGENATKPEEEDDEVKFPPIVIKTTFPEELQRFLPQGENVHTVILDFTQVNFVDSVGVKTLAGIVKEYGDVGIYVYLAGCSPQVVNDLTRNNFFENPALKELLFHSIHDAVLGSQVREAMAEQEATASLPQEDMEPNATPTTPEA

SEQ ID NO: 11 - DNA, 3848 bp, artificial sequence
  synthetic sequence
ggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtccggactcagatctctgcagaccatggatcatgctgaagaaaatgaaatccctgcagagacccagaggtactacgtggaaaggcccatcttcagtcatcctgtcctccaagagaggctgcacgtcaaggacaaagtcacagagtccattggagataagctgaagcaggcattcacgtgtactcctaaaaaaataagaaacatcatttacatgttcctgcctatcactaagtggctgccagcatataaattcaaggagtatgtgttaggtgacttggtctcgggcataagcactggggtactccagcttccccaaggcttagccttcgccatgctggcagccgtgcctccggtgtttggcctgtactcatcgttttaccccgttatcatgtactgtttctttggaacctcaagacacatatctataggtccttttgctgttattagcttgatgattggaggtgtggccgtccggttagtaccagatgatattgtcatcccaggaggagtaaatgcaaccaacgggacagaagccagagatgcactaagagtgaaagtcgccatgtctgttaccttactttcaggaatcattcagttttgcctaggtgtctgtaggtttggatttgtggccatatacctcacggagccattggtgcgaggctttaccactgcggctgctgtccacgtgttcacgtccatgttaaaatacctgtttggggtcaaaacaaagcggtacagtggaatcttttcagtggtgtatagtacagttgctgtgttgcagaatgttaaaaacctcaacgtgtgttccctaggcgtcggcctgatggtttttggtttgctgttgggtggcaaggaatttaatgagagatttaaagagaaattgccagcacccattcctctagagttctttgctgtggtgatggggactggcatttctgcaggatttaacctacatgagtcctacagtgtggatgtcgttggaacacttcctctggggctacttcctccggccaacccagacaccagcctgttccacctggtgtatgtggacgccattgccatcgccatcgttggattttcagtgacgatctccatggccaaaaccttggcaaataagcatggctaccaggttgatggcaatcaggagctcattgccttggggatatgcaactccattggatctctcttccaaaccttctcgatttcctgctccttgtctcgaagccttgttcaggaaggaactggagggaaaacacagcttgcaggttgtttggcctcgttgatgattctgttggtcatattagccaccggattcctctttgagtcgttaccccaggctgtcctttccgccattgtgatcgtcaacctgaaaggaatgttcatgcagttctcagacctgccttttttctggagaaccagcaaaatagagctgaccatctggctgaccacctttgtgtcctccctgttcctcggcttggactacggactgattaccgccgtgatcattgctctgctcacagtgatttatagaacacagagtccaagctacaaagtcctggggcagctccctgacacggatgtgtacattgacatagatgcatatgaggaggtgaaagaaattcctggaataaaaatattccaaataaatgccccaatttactatgcaaatagcgacttgtatagcagcgctttaaaaagaaagactggagtaaacccagcactcattatgggagcgagaagaaaggccatgaggaagtacgccaaggaagttggaaatgccaacgtggccaatgctactgttgtcaaagtggatgcagaagtagacggagaaaatgctacaaaacctgaagaagaggatgatgaagtcaaatttcccccaatagtcatcaaaacaacatttcctgaagagctgcagagatttttgccccagggggaaaatgtccacactgtcattctagactttacgcaggtcaattttgtggattctgttggagtgaaaactctggccgggattgtgaaagaatatggagatgttggaatttatgtatatttagcaggatgcagcccacaagttgtgaatgacctcacccgcaacaacttttttgaaaatcctgccttgaaagagcttctgttccacagtatccacgatgcagtcctgggcagccaagttcgggaggcaatggctgaacaagaagccacagcgtcacttccccaggaggatatggagcccaatgccacacccaccacccccgaggcataactcgagggggggcccggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagc

SEQ ID NO: 12 - DNA, 3848 bp, artificial sequence
  synthetic sequence
ggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtccggactcagatctctgcagaccatggatcatgctgaagaaaccgaaatccctgcagagacccagaggtactacgtggaaaggcccatcttcagtcatcctgtcctccaagagaggctgcacgtcaaggacaaagtcacagagtccattggagataagctgaagcaggcattcacgtgtactcctaaaaaaataagaaacatcatttacatgttcctgcctatcactaagtggctgccagcatataaattcaaggagtatgtgttaggtgacttggtctcgggcataagcactggggtactccagcttccccaaggcttagccttcgccatgctggcagccgtgcctccggtgtttggcctgtactcatcgttttaccccgttatcatgtactgtttctttggaacctcaagacacatatctataggtccttttgctgttattagcttgatgattggaggtgtggccgtccggttagtaccagatgatattgtcatcccaggaggagtaaatgcaaccaacgggacagaagccagagatgcactaagagtgaaagtcgccatgtctgttaccttactttcaggaatcattcagttttgcctaggtgtctgtaggtttggatttgtggccatatacctcacggagccattggtgcgaggctttaccactgcggctgctgtccacgtgttcacgtccatgttaaaatacctgtttggggtcaaaacaaagcggtacagtggaatcttttcagtggtgtatagtacagttgctgtgttgcagaatgttaaaaacctcaacgtgtgttccctaggcgtcggcctgatggtttttggtttgctgttgggtggcaaggaatttaatgagagatttaaagagaaattgccagcacccattcctctagagttctttgctgtggtgatggggactggcatttctgcaggatttaacctacatgagtcctacagtgtggatgtcgttggaacacttcctctggggctacttcctccggccaacccagacaccagcctgttccacctggtgtatgtggacgccattgccatcgccatcgttggattttcagtgacgatctccatggccaaaaccttggcaaataagcatggctaccaggttgatggcaatcaggagctcattgccttggggatatgcaactccattggatctctcttccaaaccttctcgatttcctgctccttgtctcgaagccttgttcaggaaggaactggagggaaaacacagcttgcaggttgtttggcctcgttgatgattctgttggtcatattagccaccggattcctctttgagtcgttaccccaggctgtcctttccgccattgtgatcgtcaacctgaaaggaatgttcatgcagttctcagacctgccttttttctggagaaccagcaaaatagagctgaccatctggctgaccacctttgtgtcctccctgttcctcggcttggactacggactgattaccgccgtgatcattgctctgctcacagtgatttatagaacacagagtccaagctacaaagtcctggggcagctccctgacacggatgtgtacattgacatagatgcatatgaggaggtgaaagaaattcctggaataaaaatattccaaataaatgccccaatttactatgcaaatagcgacttgtatagcagcgctttaaaaagaaagactggagtaaacccagcactcattatgggagcgagaagaaaggccatgaggaagtacgccaaggaagttggaaatgccaacgtggccaatgctactgttgtcaaagtggatgcagaagtagacggagaaaatgctacaaaacctgaagaagaggatgatgaagtcaaatttcccccaatagtcatcaaaacaacatttcctgaagagctgcagagatttttgccccagggggaaaatgtccacactgtcattctagactttacgcaggtcaattttgtggattctgttggagtgaaaactctggccgggattgtgaaagaatatggagatgttggaatttatgtatatttagcaggatgcagcccacaagttgtgaatgacctcacccgcaacaacttttttgaaaatcctgccttgaaagagcttctgttccacagtatccacgatgcagtcctgggcagccaagttcgggaggcaatggctgaacaagaagccacagcgtcacttccccaggaggatatggagcccaatgccacacccaccacccccgaggcataactcgagggggggcccggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagc

SEQ ID NO: 13 - DNA, 3379 bp, artificial sequence
  synthetic sequence
ggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtccggactcagatctctgcagaccatggatcatgctgaagaaaatgaaatccctgcagagacccagaggtactacgtggaaaggcccatcttcagtcatcctgtcctccaagagaggctgcacgtcaaggacaaagtcacagagtccattggagataagctgaagcaggcattcacgtgtactcctaaaaaaataagaaacatcatttacatgttcctgcctatcactaagtggctgccagcatataaattcaaggagtatgtgttaggtgacttggtctcgggcataagcactggggtactccagcttccccaaggcttagccttcgccatgctggcagccgtgcctccggtgtttggcctgtactcatcgttttaccccgttatcatgtactgtttctttggaacctcaagacacatatctataggtccttttgctgttattagcttgatgattggaggtgtggccgtccggttagtaccagatgatattgtcatcccaggaggagtaaatgcaaccaacgggacagaagccagagatgcactaagagtgaaagtcgccatgtctgttaccttactttcaggaatcattcagttttgcctaggtgtctgtaggtttggatttgtggccatatacctcacggagccattggtgcgaggctttaccactgcggctgctgtccacgtgttcacgtccatgttaaaatacctgtttggggtcaaaacaaagcggtacagtggaatcttttcagtggtgtatagtacagttgctgtgttgcagaatgttaaaaacctcaacgtgtgttccctaggcgtcggcctgatggtttttggtttgctgttgggtggcaaggaatttaatgagagatttaaagagaaattgccagcacccattcctctagagttctttgctgtggtgatggggactggcatttctgcaggattttccctacatgagtcctacagtgtggatgtcgttggaacacttcctctggggctacttcctccggccaacccagacaccagcctgttccacctggtgtatgtggacgccattgccatcgccatcgttggattttcagtgacgatctccatggccaaaaccttggcaaataagcatggctaccaggttgatggcaatcaggagctcattgccttggggatatgcaactccattggatctctcttccaaaccttctcgatttcctgctccttgtctcgaagccttgttcaggaaggaactggagggaaaacacagcttgcaggttgtttggcctcgttgatgattctgttggtcatattagccaccggattcctctttgagtcgttaccccaggctgtcctttccgccattgtgatcgtcaacctgaaaggaatgttcatgcagttctcagacctgccttttttctggagaaccagcaaaatagagctgaccatctggctgaccacctttgtgtcctccctgttcctcggcttggactacggactgattaccgccgtgatcattgctctgctcacagtgatttatagaacacagagtccaagctacaaagtcctggggcagctccctgacacggatgtgtacattgacatagatgcatatgaggaggtgaaagaaattcctggaataaaaatattccaaataaatgccccaatttactatgcaaatagcgacttgtatagcagcgctttaaaaagaaagactggagtaaacccagcactcattatgggagcgagaagaaaggccatgaggaagtacgccaaggaagttggaaatgccaacgtggccaatgctactgttgtcaaagtggatgcagaagtagacggagaaaatgctacaaaacctgaagaagaggatgatgaagtcaaatttcccccaatagtcatcaaaacaacatttcctgaagagctgcagagatttttgccccagggggaaaatgtccacactgtcattctagactttacgcaggtcaattttgtggattctgttggagtgaaaactctggccgggattgtgaaagaatatggagatgttggaatttatgtatatttagcaggatgcagcccacaagttgtgaatgacctcacccgcaacaacttttttgaaaatcctgccttgaaagagcttctgttccacagtatccacgatgcagtcctgggcagccaagttcgggaggcaatggctgaacaagaagccacagcgtcacttccccaggaggatatggagcccaatgccacacccaccacccccgaggcataactcgagggggggcccggtaccgcgggcccgggatccaccggatctagataactgatcataatcagccataccacatttgtagaggttttacttgctttaaaaaacctcccacacctc

SEQ ID NO: 14 - DNA, 3705 bp, artificial sequence
  synthetic sequence
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtccggactcagatctctgcagaccatggatcatgctgaagaaaccgaaatccctgcagagacccagaggtactacgtggaaaggcccatcttcagtcatcctgtcctccaagagaggctgcacgtcaaggacaaagtcacagagtccattggagataagctgaagcaggcattcacgtgtactcctaaaaaaataagaaacatcatttacatgttcctgcctatcactaagtggctgccagcatataaattcaaggagtatgtgttaggtgacttggtctcgggcataagcactggggtactccagcttccccaaggcttagccttcgccatgctggcagccgtgcctccggtgtttggcctgtactcatcgttttaccccgttatcatgtactgtttctttggaacctcaagacacatatctataggtccttttgctgttattagcttgatgattggaggtgtggccgtccggttagtaccagatgatattgtcatcccaggaggagtaaatgcaaccaacgggacagaagccagagatgcactaagagtgaaagtcgccatgtctgttaccttactttcaggaatcattcagttttgcctaggtgtctgtaggtttggatttgtggccatatacctcacggagccattggtgcgaggctttaccactgcggctgctgtccacgtgttcacgtccatgttaaaatacctgtttggggtcaaaacaaagcggtacagtggaatcttttcagtggtgtatagtacagttgctgtgttgcagaatgttaaaaacctcaacgtgtgttccctaggcgtcggcctgatggtttttggtttgctgttgggtggcaaggaatttaatgagagatttaaagagaaattgccagcacccattcctctagagttctttgctgtggtgatggggactggcatttctgcaggattttccctacatgagtcctacagtgtggatgtcgttggaacacttcctctggggctacttcctccggccaacccagacaccagcctgttccacctggtgtatgtggacgccattgccatcgccatcgttggattttcagtgacgatctccatggccaaaaccttggcaaataagcatggctaccaggttgatggcaatcaggagctcattgccttggggatatgcaactccattggatctctcttccaaaccttctcgatttcctgctccttgtctcgaagccttgttcaggaaggaactggagggaaaacacagcttgcaggttgtttggcctcgttgatgattctgttggtcatattagccaccggattcctctttgagtcgttaccccaggctgtcctttccgccattgtgatcgtcaacctgaaaggaatgttcatgcagttctcagacctgccttttttctggagaaccagcaaaatagagctgaccatctggctgaccacctttgtgtcctccctgttcctcggcttggactacggactgattaccgccgtgatcattgctctgctcacagtgatttatagaacacagagtccaagctacaaagtcctggggcagctccctgacacggatgtgtacattgacatagatgcatatgaggaggtgaaagaaattcctggaataaaaatattccaaataaatgccccaatttactatgcaaatagcgacttgtatagcagcgctttaaaaagaaagactggagtaaacccagcactcattatgggagcgagaagaaaggccatgaggaagtacgccaaggaagttggaaatgccaacgtggccaatgctactgttgtcaaagtggatgcagaagtagacggagaaaatgctacaaaacctgaagaagaggatgatgaagtcaaatttcccccaatagtcatcaaaacaacatttcctgaagagctgcagagatttttgccccagggggaaaatgtccacactgtcattctagactttacgcaggtcaattttgtggattctgttggagtgaaaactctggccgggattgtgaaagaatatggagatgttggaatttatgtatatttagcaggatgcagcccacaagttgtgaatgacctcacccgcaacaacttttttgaaaatcctgccttgaaagagcttctgttccacagtatccacgatgcagtcctgggcagccaagttcgggaggcaatggctgaacaagaagccacagcgtcacttccccaggaggatatggagcccaatgccacacccaccacccccgaggcataactcgagggggggcccggtaccgcgggcccgggatccaccggatctagataactgatcataatcagccataccacatttgtagaggttttacttgctttaaaaaacctcccacacctc

(19) United States
(12) Patent Application Publication
(10) Pub. No.: US 2019/0374763 A1
(43) Pub. Date: Dec. 12, 2019

(54) ULTRASOUND-SENSING PROTEINS AND METHOD FOR STIMULATING CELLS BY ULTRASOUND

(71) Applicant: National Tsing Hua University, Hsinchu (TW)

(72) Inventors: Yu-Chun LIN, Hsinchu (TW); Chih-Kuang YEH, Hsinchu (TW)

(21) Appl. No.: 16/290,214

(22) Filed: Mar. 1, 2019

(30) Foreign Application Priority Data
Jun. 12, 2018 (TW) 107120094

(51) Int. Cl. A61M 37/00; A61N 7/00; C07K 14/47

(52) U.S. Cl. CPC A61M 37/0092; A61N 7/00; A61N 2007/006; A61N 2007/0026; C07K 14/47

(57) ABSTRACT

An ultrasound-sensing protein is disclosed, which is a mutant of Prestin in cochlear outer hair cells of non-sonar mammals. The mutant of Prestin has a substitution of serine for asparagine at position 308 and selectively has a substitution of threonine for asparagine at position 7.

### CROSS REFERENCE TO RELATED APPLICATIONS

This application claims the benefits of the Taiwan Patent Application Serial Number 107120094, filed on Jun. 12, 2018, the subject matter of which is incorporated herein by reference.

### FIELD OF THE INVENTION

The present invention relates to an ultrasound-sensing protein and a method for stimulating cells by ultrasound, and more particularly to a mutant of Prestin in cochlear outer hair cells of non-sonar mammals and a method of stimulating cells expressing the ultrasound-sensing protein by ultrasound.

### DESCRIPTION OF RELATED ART

In nature, various organisms are capable of sensing distinct environmental inputs such as light, heat, chemicals and magnetic field and therefore can react to them accordingly. Specific proteins in cells are required for organisms' sensing of various stimuli, and may further influence the physiological activity of cells upon different environmental stimuli. Therefore, based on the response mechanisms, human cells can be endowed with the ability to sense the stimuli such as light, heat, chemicals and magnetic field by introduction of various specific proteins. In this way, the physiological activity of specific cells may be manipulated by different stimulation for various therapeutic purposes.

Although various proteins capable of sensing light, chemicals or magnetic field have been developed, there is still a significant limitation in therapeutic applications. For example, in case light-sensing proteins are introduced into cells of the specific human tissue, due to poor light penetration, deep cells within the body cannot be directly irradiated and thus invasive illumination is required for stimulating the specific proteins to manipulate cellular activity. However, surgery taken for invasive light delivery into the body may cause patient's physiological burden. As for the prior art of introducing the protein capable of sensing chemicals or magnetic field into cells of the specific tissue, the difficulty in cellular localization may lead to delayed or poor stimulation effect.

Thus, there is a need to develop a non-invasive method for precise cellular localization in an organism and cellular stimulation so as to achieve a purpose of precisely manipulating the physiological activity of specific cells.

### SUMMARY OF THE INVENTION

In accordance with the foregoing objectives, the present invention provides a modified protein related to high-frequency hearing in nature. The modified protein has an ability to sense an ultrasound excitation with a specific frequency. Human cells can be endowed with the ability to sense the ultrasound excitation by introduction of the modified protein thereinto or expression of the modified protein therein. Therefore, specific cells may be non-invasively stimulated by medical focused ultrasound to influence its physiological activity for therapeutic purposes.

The present invention provides an ultrasound-sensing protein, which is a mutant of Prestin in cochlear outer hair cells of non-sonar mammals. The mutant of Prestin has a substitution of serine for asparagine at position 308 and selectively has a substitution of threonine for asparagine at position 7.

In one preferred embodiment of the present invention, the ultrasound-sensing protein is a mutant of Prestin in cochlear outer hair cells of non-sonar mammals, which has a substitution of serine for asparagine at position 308 and has a substitution of threonine for asparagine at position 7.

In one embodiment of the present invention, the Prestin in cochlear outer hair cells of non-sonar mammals is a Prestin in cochlear outer hair cells of human, mouse, Pteropus vampyrus, Balaenoptera acutorostrata, Eonycteris spelaea or Rousettus leschenaultia. The Prestin sequence of cochlear outer hair cells of human is SEQ ID NO: 1. The Prestin sequence of cochlear outer hair cells of mouse is SEQ ID NO: 2. The Prestin sequence of cochlear outer hair cells of Pteropus vampyrus is SEQ ID NO: 3. The Prestin sequence of cochlear outer hair cells of Balaenoptera acutorostrata is SEQ ID NO: 4. The Prestin sequence of cochlear outer hair cells of Eonycteris spelaea is SEQ ID NO: 5. The Prestin sequence of cochlear outer hair cells of Rousettus leschenaultia is SEQ ID NO: 6.

In the present invention, the sequence of the N308S mutant of Prestin in cochlear outer hair cells of human is SEQ IDNO: 7, the sequence of the N7T and N308S mutant of Prestin in cochlear outer hair cells of human is SEQ ID NO: 8, the sequence of the N308S mutant of Prestin in cochlear outer hair cells of mouse is SEQ IDNO: 9, and the sequence of the N7T and N308S mutant of Prestin in cochlear outer hair cells of mouse is SEQ ID NO: 10.

The present invention further provides a method for stimulating cells, comprising a step of irradiating ultrasound on a cell capable of expressing an ultrasound-sensing protein. The ultrasound-sensing protein is a mutant of Prestin in cochlear outer hair cells of non-sonar mammals. The mutant of Prestin has a substitution of serine for asparagine at position 308 (N308S) and selectively has a substitution of threonine for asparagine at position 7 (N7T).

In one preferred embodiment, the ultrasound-sensing protein is a mutant of Prestin in cochlear outer hair cells of non-sonar mammals. The mutant of Prestin has a substitution of serine for asparagine at position 308 and selectively has a substitution of threonine for asparagine at position 7.

In one embodiment, the ultrasound is a focused ultrasound. In addition, a preferable frequency of the ultrasound is 0.35˜0.65 MHz, and a preferable acoustic pressure of the ultrasound is 0.1˜1.0 MPa. Also, the optimal frequency is 0.5 MHz, and the optimal acoustic pressure is 0.5 MPa.

In one embodiment of the present invention, calcium influx is induced into the cell upon ultrasound irradiation on the cell.

In one embodiment of the present invention, the cell may be a nerve cell, an immune cell, an islet cell, an epithelial cell, a blood cell, a muscle cell, a stein cell, and other eukaryotic cells.

The ultrasound-sensing protein of the present invention can sense ultrasound stimulus. By introduction of ultrasound-sensing proteins into cells or expression of ultrasound-sensing proteins in cells, ultrasound irradiation can be applied to gene regulation, neuromodulation, and immunomodulation. Accordingly, the series of sonogenetic approaches developed in the present invention can serve as new strategies to precisely manipulate cellular activities for various therapeutic applications and make a significant breakthrough in therapy.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present invention will become more readily apparent to those ordinarily skilled in the art after reviewing the following detailed description and accompanying drawings, in which:

FIG. 1 shows fluorescence images of the control cells and the cells expressing Venus-mPrestin (N7T, N308S) upon ultrasound stimulation in one embodiment of the present invention.

FIG. 2 shows the fold of probability of calcium response in various groups in one embodiment of the present invention.

FIG. 3 shows fluorescence images (observed by optical microscope) of immunohistochemical stained cells expressing Prestin upon non-invasive ultrasound stimulation.

### DETAILED DESCRIPTION OF THE PREFERRED EMBODIMENTS

### [Cells Culture]

293T cells were cultured in Dulbecco's modified Eagle's medium (DMEM, Gibco) supplemented with 10% fetal bovine serum (FBS), 5 U/mL penicillin and 50 μg/mL streptomycin.

For preparing 293T cells expressing Venus-mPrestin WT, Venus-mPrestin (N7T), Venus-mPrestin (N308S) and Venus-mPrestin (N7T, N308S), 293T cells were transfected with Venus-mPrestin (wild type) DNA (SEQ ID NO:11), Venus-mPrestin (N7T) DNA (SEQ ID NO:12), Venus-mPrestin (N308S) DNA (SEQ ID NO:13), or Venus-mPrestin (N7T, N308S) DNA (SEQ ID NO:14), respectively, with LT-1 transfection reagent (Minis).

### [Response of Cells Expressing Prestin to Different Ultrasound Frequencies]

A general cellular response to mechanical stimuli, calcium response, is used as readout upon ultrasound stimulus. Low frequency of ultrasound (<3.5 MHz, 0.5 MPa, 2000 cycles) has good penetration without inducing thermal effects or damages on the tissues. Therefore, ultrasound was used to stimulate the cells co-expressing a calcium biosensor (red), R-GECO, and Venus (yellow fluorescent protein) as control group or different Venus-mPrestin, including Venus-mPrestin WT, Venus-mPrestin (N7T), Venus-mPrestin (N308S) and Venus-mPrestin (N7T, N308S).

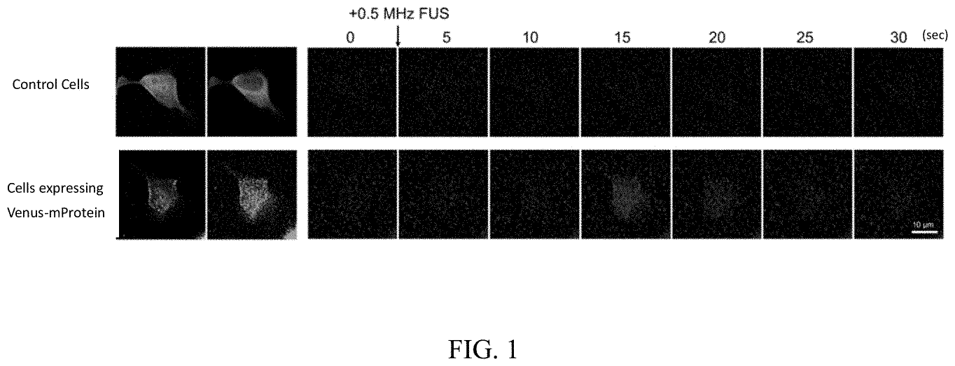

FIG. 1 shows cell imaging results of 293T cells co-transfected with a calcium biosensor (R-GECO, red) and Venus (yellow fluorescent protein) or Venus-mPrestin (N7T, N308S) by gene transfection. This experiment demonstrated that ultrasound excitation (0.5 MHz, 0.5 MPa, 2000 cycles, 3 seconds) effectively evokes calcium responses in Venus-mPresin (N7T, N308S)-transfected cells, resulting in increased red fluorescence intensity of the calcium biosensor, but not in control cells (Venus only).

In the cell fluorescence image of FIG. 1, the green, cyan and red fluorescence signals indicate distribution of Venus protein (control group) or Venus-mPrestin (N7T, N308S) protein, distribution of calcium biosensor and distribution of calcium, respectively. As shown in FIG. 1, excitation of 0.5 MHz ultrasound induces calcium responses in the cells expressing Venus-mPrestin (N7T, N308S), leading to large calcium influx into the cells. However, no calcium response was observed in the control cells upon ultrasound stimulus.

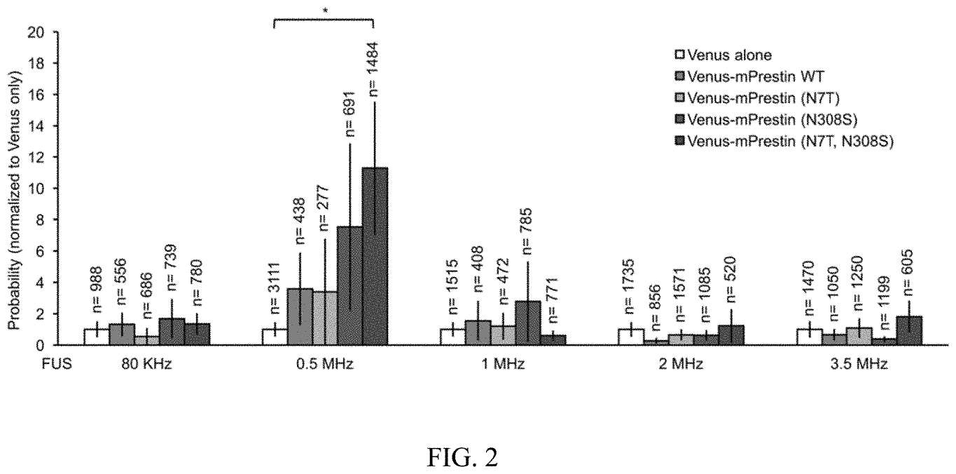

For evaluation on responses to different ultrasound frequencies, the 293T cells co-transfected with calcium biosensor (red) and Venus, Venus-mPrestin (wild type), Venus-mPrestin (N7T), Venus-mPrestin (N308S) or Venus-mPrestin (N7T, N308S) were excited by ultrasound with different frequencies (80 kHz-3.5 MHz, 0.5 MPa, 2000 cycles, 3 seconds). The percentages of calcium responding cells were calculated and divided by that of control cells. The results were shown in FIG. 2.

FIG. 2 shows the fold of probability of calcium response in various groups normalized to Venus alone control group. As shown in FIG. 2, significant calcium response is observed in Venus-mPresin (N308S)-transfected cells and Venus-mPresin (N7 T, N308S)-transfected cells upon 0.5 MHz ultrasound stimulus. Particularly, the Venus-mPresin (N7T, N308S)-transfected cells has ˜11 folds better calcium response compared to control cells, exhibiting a significant difference (p<0.05).

As shown in the above results, cells expressing mPrestin (N7T, N308S) are highly sensitive to 0.5 MHz ultrasound stimulation, and the percentage of ultrasound responding cells is ˜11 folds higher than control cells. These results demonstrated that mPrestin (N7T, N308S) endows transfected mammalian cells with the ability to sense 0.5 MHz ultrasound stimulation.

### [Non-Invasive Stimulation on Cells Expressing Prestin]

All protocols involving animals were approved by the National Tsing-Hua University animal committee (IACUC approval number: NTHU10459).

In this experiment, neuronal cells in the mouse brain were transfected with DNA fragments for expressing Venus-mPrestin (N7T, N308S), followed by ultrasound stimulation on neuronal cells expressing mPrestin (N7T, N308S) to test whether ultrasound can stimulate the activity of neuronal cells in mouse brains with intact skull. The detailed experimental procedure was as follows.

1,2-dipalmitoyl-sn-glycero-3-phosphocholine (DPPC, Avanti Polar Lipids, AL, USA), 1,2-dipalmitoyl-3-trimethylammonium-propane (DPTAP, Avanti Polar Lipids), and 1,2-distearoyl-sn-glycero-3-phospho ethanolamine-N-[carboxy(polyethylene glycol)-2000] (DSPE-PEG 2K, Avanti Polar Lipids) (molar ratio of 31.5:3.9:1.8) were dissolved in chloroform and dried over 24 hs. The dried lipid film was then mixed with glycerol-PBS (5 μL/mL) with C₃F₈ gas and shaken in an agitator for 45 s to form microbubbles (MBs). Then, the unreacted lipids were removed from MBs via centrifugation (2 mins, 6,000 rpm). The cationic property of DPTAP enables spontaneous attachment to the plasmid by electrostatic interaction.

For preparation of pPrestin-loaded cMBs (pPrestin-cMBs), 5 μg of pPrestin (Venus-Prestin DNA) was mixed with MBs (2×10⁸ MBs/μL, 50 μL), gently rotated for 30 mins, and then centrifuged (2 mins, 6,000 rpms) to separate unloaded pPrestin from well-conjugated pPrestin-MBs. The successfully binding of DNA onto the lipid shell of MBs was imaged via propidine iodide staining with microscopy. The DNA loading efficiency of pPrestin-MBs was evaluated by the spectrophotometer as follows:

$$DNA loading efficiency ( \% ) = \frac{weight of pPrestin loaded on {10}^{9} cMBs}{total weight of pPrestin added in {10}^{9} cMBs} \times 100 \%$$

The experimental result showed that the DNA loading efficiency is 24.5±1.6%, and the practical DNA loading amount of the microbubbles is 1.2±0.1 μg.

Further, pVenus-MBs as comparison group were prepared by the same method as pPrestin-MBs, with adding pVenus (Venus DNA).

Prior to the below cell transfection, male C57BL/6JNarl mice (N=9, 6-10 weeks in age) were anesthetized with isoflurane gas (dose: 1%; flow rate: 1 L/min) and pure oxygen.

In vivo gene transfection was conducted by a 1-MHz focused ultrasound (FUS) transducer (V302, Panametrics, Waltham, Mass., USA; diameter=38 mm, focus length=60 mm) with pPrestin-MBs.

The animals were randomly divided into two groups: pPrestin-MBs combined with FUS (pPrestin-MBs+FUS) as experimental group and pVenus-MBs combined with FUS (pVneus-MBs+FUS) as comparison group. Mice were infused with pPrestin-cMBs by retro-orbital injection. Waiting 20 seconds, FUS sonication was applied transcranially in the left hemisphere of the brain at 0.5 MPa peak-rarefactional acoustic pressure with 10,000 of cycle, 5 Hz of pulse repetition frequency, and two sites of sonication, resulting in Blood Brain Barrier (BBB)-opening for delivery of DNA-carrying microbubbles to cells for gene transfection.

At 48 hrs after gene transfection, in vivo neuronmodulation was conducted by a 0.5-MHz FUS transducer (V389, Panametrics) (2,000 cycle, pulse repetition frequency of 1 Hz, one sites of sonication, and duration of 3 s of sonication per site). Normal mice (N=3) without pPrestin transfection were also received 0.5-MHz ultrasound for comparison.

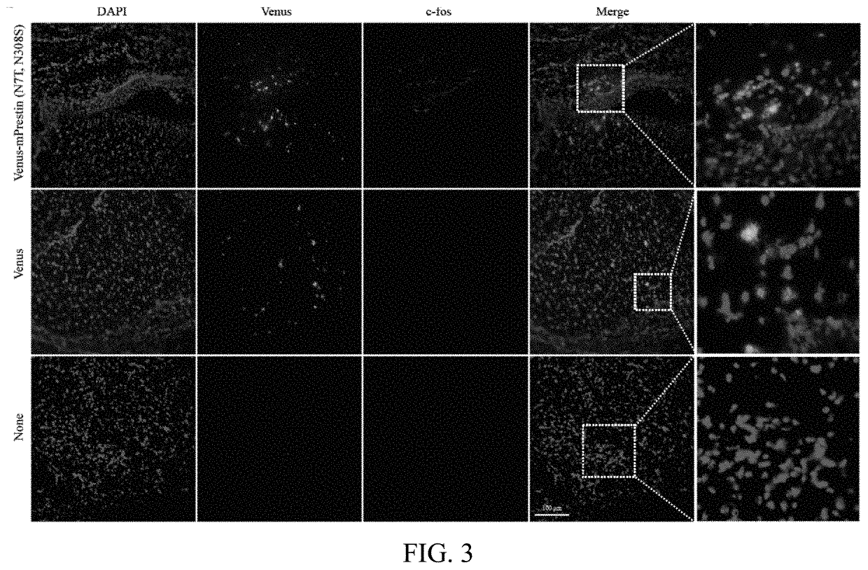

After 0.5-MHz FUS stimulation, the brains of mice were removed and sliced into 15-μm sections. The sections were fixed in −20° C. methanol for 20 mins, and endogenous proteins were blocked by incubation in a solution of 5% goat serum and 1% BSA with PBS. The sections were then incubated in primary rabbit anti-c-Fos antibody (1:1000) in antibody diluent for overnight. The sections were then incubated for 1 h in Dylight 594 conjugated anti-rabbit secondary antibody (1:200) in antibody diluent followed by several washes in PBS. The cellular nuclei were labelled by DAPI. Finally, the slides were coverslipped with fluorescent mounting medium and stored flat in the dark at −20° C. Evaluation of the immunohistochemical staining was performed by light microscope. As shown in FIG. 3, the successful transfection of pPrestin and pVenus was confirmed by the expression of VENUS fluorescence protein (green), and activated neuronal cells were labeled by c-fos antibody (red).

The above experiments demonstrated that extracorporeal ultrasound irradiation can manipulate activities of cells transfected with ultrasound-sensing proteins.

What is claimed is:

1. An ultrasound-sensing protein, which is a mutant of Prestin in cochlear outer hair cells of non-sonar mammals, wherein the mutant of Prestin has a substitution of serine for asparagine at position 308 (N308S) and optionally has a substitution of threonine for asparagine at position 7 (N7T).

2. The ultrasound-sensing protein according to claim 1, wherein the Prestin in cochlear outer hair cells of non-sonar mammals is a Prestin of cochlear outer hair cells of human, mouse, Pteropus vampyrus, Balaenoptera acutorostrata, Eonycteris spelaea or Rousettus leschenaultia.

3. The ultrasound-sensing protein according to claim 2, wherein an amino acid sequence of the Prestin in cochlear outer hair cells of human is SEQ ID NO: 1, an amino acid sequence of the Prestin in cochlear outer hair cells of mouse is SEQ ID NO: 2, an amino acid sequence of the Prestin of cochlear outer hair cells of Pteropus vampyrus is SEQ ID NO: 3, an amino acid sequence of the Prestin of cochlear outer hair cells of Balaenoptera acutorostrata is SEQ ID NO: 4, an amino acid sequence of the Prestin of cochlear outer hair cells of Eonycteris spelaea is SEQ ID NO: 5, and an amino acid sequence of the Prestin of cochlear outer hair cells of Rousettus leschenaultia is SEQ ID NO: 6.

4. The ultrasound-sensing protein according to claim 3, wherein an amino acid sequence of the N308S mutant of Prestin in cochlear outer hair cells of human is SEQ IDNO: 7, an amino acid sequence of the N7T and N308S mutant of Prestin in cochlear outer hair cells of human is SEQ ID NO: 8, an amino acid sequence of the N308S mutant of Prestin in cochlear outer hair cells of mouse is SEQ IDNO: 9, and an amino acid sequence of the N7T and N308S mutant of Prestin in cochlear outer hair cells of mouse is SEQ ID NO: 10.

5. A method for stimulating cells, comprising a step of irradiating ultrasound on a cell capable of expressing an ultrasound-sensing protein, wherein the ultrasound-sensing protein is a mutant of Prestin in cochlear outer hair cells of non-sonar mammals, and the mutant of Prestin has a substitution of serine for asparagine at position 308 (N308S) and optionally has a substitution of threonine for asparagine at position 7 (N7T).

6. The method according to claim 5, wherein the ultrasound is a focused ultrasound.

7. The method according to claim 6, wherein a frequency of the ultrasound is 0.5 MHz, and an acoustic pressure of the ultrasound is 0.5 MPa.

8. The method according to claim 5, wherein calcium influx is induced into the cell upon ultrasound irradiation on the cell.

9. The method according to claim 5, wherein the cell is a nerve cell, an immune cell, an islet cell, an epithelial cell, a blood cell, a muscle cell, a stein cell or any other eukaryotic cell.
